(19) United States
(12) Patent Application Publication
(10) Pub. No.: US 2026/0285711 A1
(43) Pub. Date: Sep. 24, 2026

(54) UV-LED ASSEMBLY WITH PCB SUBSTRATE INTEGRATED INTO HOUSING

(71) Applicant: TROJAN TECHNOLOGIES GROUP ULC, London (CA)

(72) Inventors: David A. OLSON, London (CA); Michael SASGES, London (CA)

(73) Assignee: TROJAN TECHNOLOGIES GROUP ULC, London (CA)

(21) Appl. No.: 19/546,614

(22) Filed: Feb. 23, 2026

Related U.S. Application Data
(60) Provisional application No. 63/774,362, filed on Mar. 19, 2025.

(51) Int. Cl. C02F 1/32; A61L 2/10

(52) U.S. Cl. CPC C02F 1/325; A61L 2/10; A61L 2202/11; A61L 2202/121; A61L 2202/122; C02F 2201/004; C02F 2201/3222; C02F 2201/3225; C02F 2201/3227; C02F 2303/04

(57) ABSTRACT

This disclosure provides a UV-LED assembly and a reactor that incorporates the UV-LED assembly to treat fluid with UV light. The UV-LED assembly includes a printed circuit board with a plurality of UV-LEDs and a metal core or ceramic core substrate, and a transparent window arranged on the UV-LED assembly so that UV light from the UV-LEDs passes through the transparent window. The assembly is formed so that a surface of the metal core or ceramic core substrate constitutes an exterior surface of the UV-LED assembly. When used in the reactor, the fluid or a separate cooling fluid can contact the exterior surface to dissipate heat generated by the LEDs.

### CROSS-REFERENCE TO RELATED APPLICATIONS

This application claims the earlier filing date benefit of U.S. Provisional Application No. 63/774,362, filed on Mar. 19, 2025, the entirety of which is incorporated by reference herein.

### TECHNICAL FIELD

This application relates generally to UV-LED assemblies with improved heat dissipation, and more particularly, to UV-LED assemblies in which LEDs are supported on a metal or ceramic core printed circuit board, and the metal core or ceramic core is integrated into the assembly housing and constitutes an exterior surface of the housing. The UV-LED assemblies are useful in reactors that treat or disinfect fluids with UV light and where the fluid flowing through the reactor can also cool the UV-LED assembly.

### BACKGROUND

Disinfection of water is critical to ensure water quality. Water sources can be contaminated with pathogens, such as bacteria, viruses, fungi, algae, molds, and yeasts, making the water unsafe for consumption or use by humans and animals. One way of disinfecting water is by ultraviolet (UV) radiation treatment, in which water is irradiated with UV light. UV radiation damages the DNA, RNA, and protein in pathogens, and inactivates them, making the water safe for use and consumption. UV radiation treatment can be used in residential, municipal, commercial, and industrial water systems.

Conventional UV radiation treatment systems vary depending on the application, but in general, fluid flows through a treatment chamber of a reactor and is exposed to UV light from a UV light source. Different UV light sources can be used. For example, large industrial and commercial systems may include a large reactor including multiple UV lamps, such as mercury lamps, for effectively disinfecting a large volume of water. More recently, multiple UV light-emitting diodes (LEDs) can be used as the UV light source.

Currently, a significant amount of the energy that is supplied to LEDs is discharged as heat, and the heat must be adequately dissipated to the surrounding environment so that the LEDs can operate with the output that is required to disinfect the fluid without overheating. There are several technological challenges in providing adequate heat transfer to cool the LEDs in these systems.

### SUMMARY

One way of dissipating heat from LEDs is to design the reactor so that the treatment fluid, or a separate cooling fluid, contacts an assembly that contains the LEDs. The assembly can include a metal housing that holds the LEDs and a quartz window that allows the UV light to be transmitted to the fluid. In such designs, the LEDs have been provided on a metal core printed circuit board (“MCPCB”) that is contained in the housing. MCPCBs contain a base metal material (e.g., copper, aluminum, stainless steel) that dissipates heat generated by the LEDs. The MCPCB is placed in the housing so that the backside of the metal core substrate is in direct contact with the metal housing of the assembly or with a layer of thermal interface material (TIM) that is interspersed therebetween. In connection with this disclosure, it has been discovered that this arrangement has significant thermal resistance that is caused by decreased surface-to-surface contact due to surface imperfections on the metal surfaces, a relatively low heat transfer coefficient of the TIM layer, and the combined thicknesses of the housing wall, TIM layer, and metal core layer.

The present disclosure takes a novel approach of using the core layer of the PCB as at least part of the assembly housing wall so that the core layer directly contacts fluid that is being treated or otherwise contacts a cooling fluid. This can eliminate or improve the above-described sources of thermal resistance, and can more effectively cool the LEDs, allow the LEDs to operate at a higher power, and prevent the LEDs from overheating.

In accordance with one aspect, this disclosure provides a reactor for treating a fluid with UV radiation. The reactor includes (i) a reactor vessel through which the fluid flows; and (ii) a UV-LED assembly that is arranged in the reactor vessel. The UV-LED assembly includes (a) a printed circuit board that includes a plurality of UV-LEDs and a metal core or ceramic core substrate, and (b) a transparent window arranged on the UV-LED assembly so that UV light from the UV-LEDs passes through the transparent window and into the fluid flowing through the reactor vessel. And a surface of the metal or ceramic core substrate constitutes a surface of the UV-LED assembly that is configured to be in direct contact with a cooling fluid.

In accordance with another aspect, this disclosure provides a UV-LED assembly that includes (a) a printed circuit board that includes a plurality of UV-LEDs and a metal core or ceramic core substrate, and (b) a transparent window arranged on the UV-LED assembly so that UV light from the UV-LEDs passes through the transparent window. And a surface of the metal core or ceramic core substrate constitutes an exterior surface of the UV-LED assembly.

### BRIEF DESCRIPTION OF THE DRAWINGS

FIG. 1 is a perspective view of a UV-LED reactor housing.

FIG. 2 is a cross-sectional view of the reactor with two UV-LED assemblies.

FIG. 3 is a front view of a UV-LED assembly.

FIG. 4 is a schematic diagram of a cross-section of an LED array on a metal core or ceramic core PCB.

FIGS. 5A and 5B are schematic diagrams of cross-sections of LED arrays on different metal core or ceramic core PCBs.

FIG. 6 is a cross-sectional perspective view of a UV-LED assembly associated with related art.

FIG. 7 is a cross-sectional perspective view of a UV-LED assembly according to one embodiment of the invention.

FIG. 8 is a cross-sectional perspective view of a UV-LED assembly according to another embodiment of the invention.

FIG. 9 is a cross-sectional view of a fluid treatment reactor with a tube-shaped UV-LED assembly.

FIG. 10 is a perspective view of a fluid treatment reactor with a cylindrical UV-LED assembly.

FIG. 11 is a schematic diagram of a cross-section of an LED array on a metal core or ceramic core PCB that is shown contacting a cooling fluid.

### DETAILED DESCRIPTION

In the following description, numerous details are set forth to provide an understanding of the present disclosure. However, it may be understood by those skilled in the art that the apparatus of the present disclosure may be practiced without these details and that numerous variations or modifications from the described embodiments may be possible. Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

UV light is an effective mechanism for killing or incapacitating microbes in fluids including but not limited to water and wastewater. UV-LED arrays are often used in industrial water systems and smaller household treatment systems to treat water. In these systems, water enters the reactor and is subjected to UV light in a treatment chamber. In such reactors, the UV-LED array is often included in an assembly that is at least partially submerged in the water. This arrangement allows the heat that is generated in the LED array to be conducted to the water and transferred to the water. In other cases, the LED assembly has a separate cooling water supply that is brought in contact with a portion of the assembly to remove heat. The assembly can include a light source unit with housing walls and a transparent window that form a water-tight cavity that houses the UV-LED array and allows the UV light to pass into the fluid. The UV-LED array is typically supported on a metal core printed circuit board (MCPCB) that is placed in the water-tight assembly.

In connection with this disclosure, the inventors have developed a UV-LED assembly with improved heat dissipation. The UV-LED assembly includes a MCPCB or ceramic core printed circuit board (“CCPCB”) with a metal core or ceramic core layer that has a surface that constitutes at least a portion of an exterior surface of the UV-LED assembly. For example, as described in detail below, the metal core or ceramic core layer/substrate can constitute a housing wall of the light source unit that houses the LED array. The UV-LED assembly can be used to treat or disinfect fluid with UV light, and the fluid being treated, or a separate cooling fluid, can directly contact the exterior surface to cool the UV-LED assembly. Integrating the core layer of the MCPCB or CCPCB into the housing wall of the UV-LED assembly improves heat transfer because there is no interface between the core layer and a separate housing wall that imparts thermal resistance, as discussed above, and there is no need for any intervening thermal interface material (TIM) that can also impart thermal resistance.

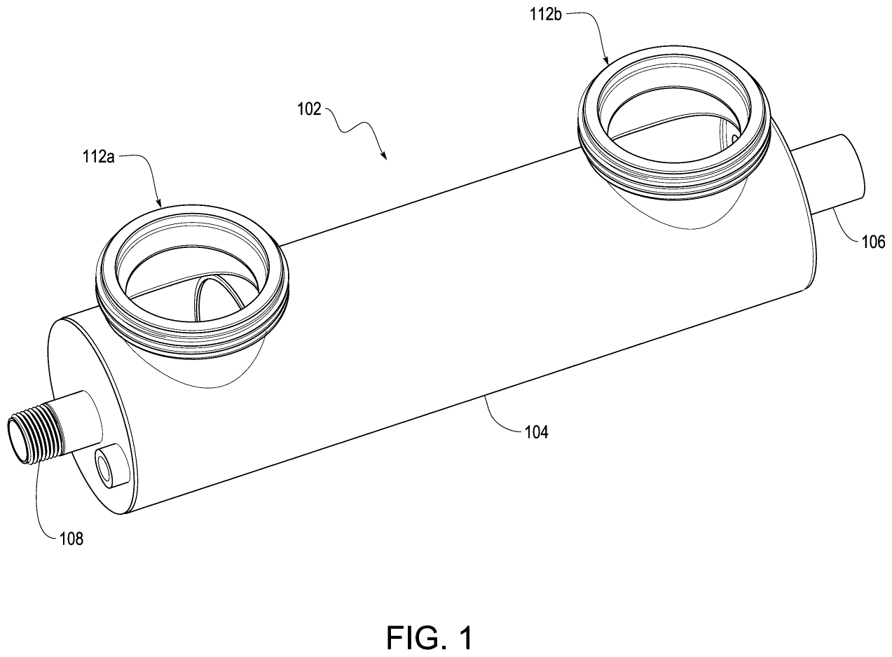

For context, FIGS. 1-3 provide a general description of a UV reactor that employs a UV-LED assembly with two puck-shaped light source units. As explained in more detail below, the UV-LED assembly can have a light source unit in a variety of shapes, including but not limited to a puck shape, a rod or cylinder shape, and a tube design, and likewise the reactor can have only a single UV-LED assembly or can have more than two UV-LED assemblies.

FIG. 1 illustrates a tubular reactor vessel 102 that can be used with two puck-shaped UV-LED assemblies. The reactor vessel includes an inlet 106 for receiving fluid to be treated in a treatment chamber within exterior wall 104 and an outlet 108 for discharging treated fluid. The outer wall 104 includes openings 112a, 112b which can each receive corresponding UV-LED assemblies, which can emit UV light into fluid that flows through the reactor vessel 102.

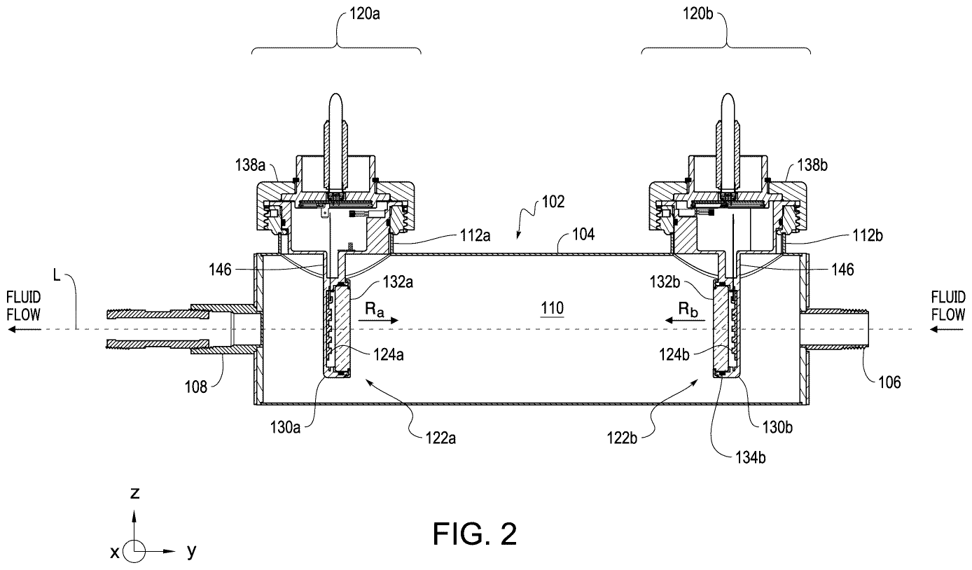

FIG. 2 is a cross-sectional view of the reactor vessel 102 with two puck-LED assemblies 120a, 120b that have been secured in openings 112a, 112b. The LED assemblies 120a, 120b are removably coupled to the reactor vessel 102 via the caps 138a, 138b such that the light source units 122a, 122b are arranged inside the treatment chamber 110 and are oriented for directing UV radiation along directions Rₐ and R_{b} into the fluid flowing through the treatment chamber 110. The fluid enters the treatment chamber 110 through inlet 106 and is treated by UV light that is emitted from UV-LED arrays 124a, 124b that are contained within the LED assemblies 120a, 120b.

The UV-LED assemblies 120a, 120b have puck-shaped light source units 122a, 122b that each are connected to the end of a mounting arm 146. The light source units 122a, 122b have metal housings 130a, 130b and transparent emission faces (i.e., transparent windows 132a, 132b). The light source units 122a, 122b are arranged so that the transparent windows 132a, 132 b face each other and emit UV light in a longitudinal direction L of the reactor vessel 102, which can increase the UV exposure of the fluid as it flows through the reactor. However, the present disclosure is not limited to this, and the light source units 122a, 122b can be arranged in any suitable orientation for sufficiently treating the fluid flowing through the treatment chamber 110 with UV radiation.

The light source units 122a, 122b are arranged in the treatment chamber 110 so as to be immersed in the fluid flowing through the treatment chamber 110 for UV treatment. For example, the light source units 122a, 122b may be arranged so as to be partially or fully immersed in the fluid flowing through the treatment chamber 110. In other words, the fluid flowing through the treatment chamber 110 impinges on and flows around the light source units 122a, 122b. The fluid not only impinges on the front, light-emitting side of the light source units 122a, 122b, but also impinges on, contacts, and flows around the back side of the light source units 122a, 122b. Thus, the fluid being treated can be used to continuously cool the light source units 122a, 122b by directly contacting the metal housing 130a, 130b.

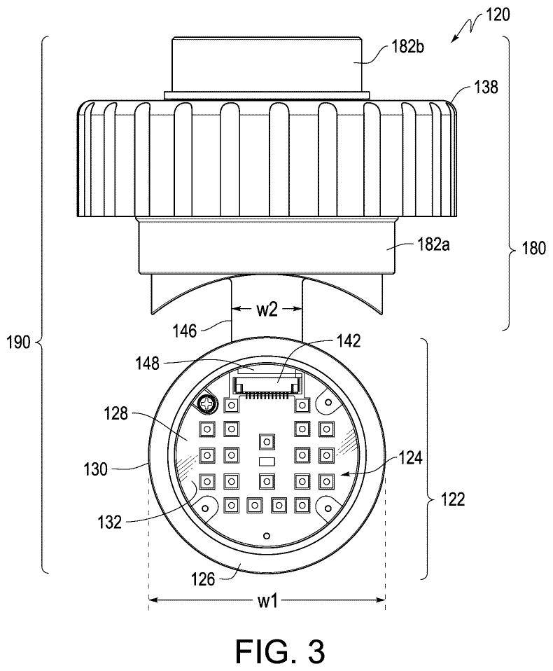

FIG. 3 illustrates additional details of the light source assembly 120. The light source assembly 120 can include a housing unit 190 including a light source unit 122, mounting arm 146 and base housing portion 180. FIG. 3 shows LED array 124 with LEDs that are mounted on and electrically coupled to printed circuit board 128, which is a CCPCB or a MCPCB. The printed circuit board 128 is arranged in metal housing 130, which may be at least partially formed from the metal core or ceramic core substrate of the CCPCB or the MCCPB, as explained below. A quartz window 132 can be secured to the housing 130 with a retaining ring 126 that is positioned over the quartz window 132 and screws onto the metal housing 130, or alternatively is snap fit into a groove in the metal housing 130. The cavity inside the metal housing 130 is water-tight so that water or other fluid cannot damage the LEDs or the corresponding circuitry. One or more cables 148 can extend through the mounting arm 146 and connect to connector 142 on the printed circuit board 128 to transmit power and/or communications signals to the light source unit 122.

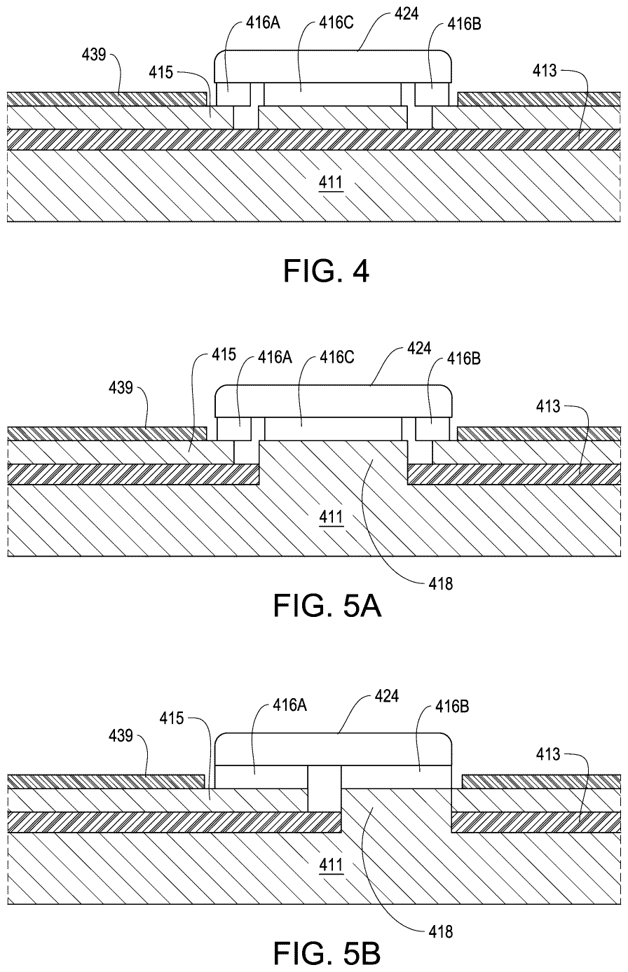

FIGS. 4, 5A, and 5B are schematic diagrams that illustrate the general structure of different MCPCBs or CCPCBs. These general structures can be employed in all embodiments described herein. FIG. 4 depicts a regularly configured MCPCB or CCPCB, and FIGS. 5A and 5B disclose a MCPCB or CCPCB that incorporates a thermal via to provide a direct thermal path through the PCB without an intervening dielectric insulation layer. Referring to these figures, both PCBs include a core layer 411 that is either a thermally conductive metal for MCPCBs (e.g., aluminum, copper, or alloys thereof, or stainless steel) or a thermally conductive ceramic for CCPCBs (e.g., alumina, aluminum nitride, beryllium oxide). The core layer 411 is a single layer (i.e., it does not comprise a laminate or composite of two or more separate layers that are stacked or joined together at an interface) with a substantially uniform composition. The core layer 411 can have a thickness in a range of from 0.25 mm to 5 mm, from 0.5 mm to 3 mm, or from 0.75 mm to 2.5 mm, for example. Each PCB includes a dielectric layer 413 that is formed directly on an upper surface of the core layer 411. The dielectric layer 413 is formed of a dielectric material that is electrically insulating. Typical dielectric materials include Dupont Coolam™ and Arlon 92ML. The dielectric layer 413 is typically less than a third of the thickness of the core layer, or even less than a fourth or a fifth of the thickness of the core layer. A typical dielectric layer thickness is between 0.1 mm and 0.2 mm. A trace layer 415 that is formed of a conductive metal such as copper can be formed on an upper surface of the dielectric layer 413. The trace layer 415 has traces that can provide an electrical connection between devices on the circuit board and can also be used to conduct heat laterally. The trace layer 415 can be less than a fifth of the thickness of the core layer 411 or less than a tenth of the thickness of the core layer, for example. A solder mask layer 439 is formed on an upper surface of the trace layer 415. Solder areas/pads 416A, 416B form the anode and cathode of the LED 424 and are used to electrically connect the trace layer 415 to the LED 424. Other layers can be included in MCPCB or CCPCP that are not illustrated, such as a top surface finish layer (Organic Solderability Preservative) and an overlay layer, for example.

In the FIG. 4 arrangement, the LED includes a thermal solder pad 416C. Thermal pad 416C is in contact with the LED 424, and the heat generated by LED 424 is dissipated through the solder pad 416C, the trace layer 415, the dielectric layer 413, and the core layer 411. In FIG. 4, all thermal paths from the LED to the core must pass through the dielectric layer 413. This layer has a significant thermal resistance, which impedes the flow of heat from the LED to the core. The arrangement shown in FIG. 5A includes a thermal via or pillar 418 that protrudes from and is integrally formed with the core layer 411 that contacts solder pad 416C, which increases heat dissipation from the LED since the heat is not transferred through a dielectric layer. The arrangement in FIG. 5B includes a thermal via or pillar 418 that protrudes from and is integrally formed with the core layer 411 and contacts solder pad 416B, which increases heat dissipation from the LED. The FIG. 5B arrangement does not include a central solder pad 416C.

For CCPCBs, it may also be possible to omit a separate dielectric layer 413 where the ceramic in the core is sufficiently electrically insulating, which would further improve heat dissipation from the LED 424. As explained in greater detail below, in embodiments of the invention the core layer 411 can be configured to form at least a portion of the wall of the LED assembly, e.g., so that one or more or a top surface, a bottom surface, or a side surface (i.e., edge) of the core layer 411, or portions of such surfaces, can directly contact a cooling fluid to provide cooling and dissipate the heat generated by the LED 424.

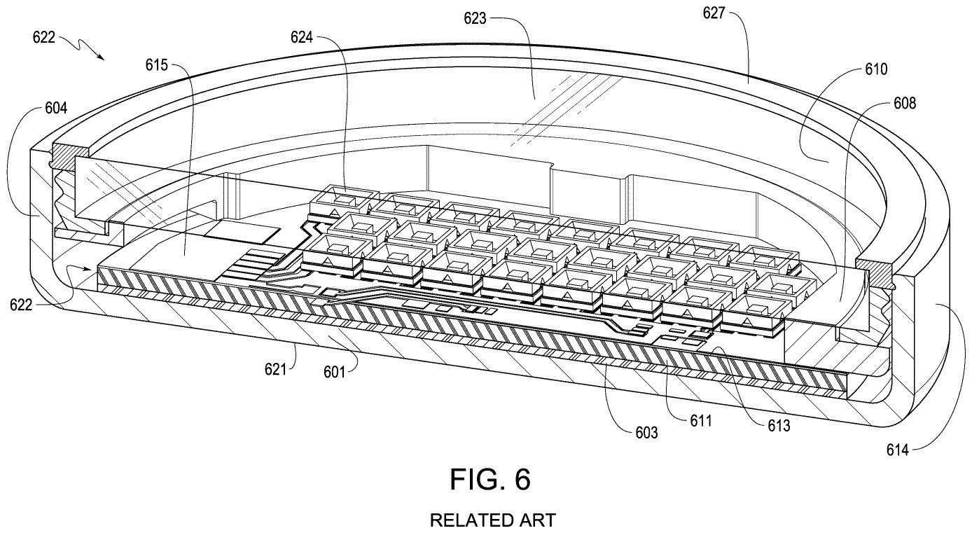

For purposes of comparison, a conventional UV-LED assembly is illustrated in FIG. 6, which is a cross-sectional view taken through the center of light source unit 622. The light source unit 622 includes a metal housing 614 that includes bottom wall 601 and sidewall 604. A layer of TIM paste 603 is provided on an inner surface of the rear wall 601. An MCPCB or conventional circuit board 622 is provided in the housing 614 in contact with the TIM paste 603. The MCPCP 622 includes metal core layer 611, a thin dielectric layer 613, and a copper trace layer 615 on top of the dielectric layer. A plurality of UV-LEDs 624 are supported on the MCPB 622 and are electrically connected to the copper trace layer 615. The light source unit 622 includes plastic bushing 608 and sealing ring 610 that support a transparent quartz window 623 that is clamped in place with a retaining ring 627. The sealing ring 610 can be made of an elastomer. A conventional O-ring could also be used to provide a seal, either on a radial surface of the metal housing 614 or on a flat face of the quartz window 623.In this figure, the retaining ring 627 snap fits into a groove that is formed on an inner circumferential surface of the housing 614, but it is also possible to use a threaded engagement between the sealing ring and the housing, and the sealing ring could also be engaged on the outer circumferential surface or the top surface of the housing 614 in other embodiments. In FIG. 6, when the light source unit 622 is installed in a reactor, fluid being treated will contact at least the housing exterior surface 621 to cool the UV-LED assembly. The heat is dissipated from the LEDs 624 through the dielectric layer 613 (or through a pillar as shown in FIG. 5), the core layer 611, the TIM layer 603, and through the housing rear wall 601. As indicated above, in this type of arrangement, heat transfer is hindered not only by the overall thickness of the various layers, but due to the fact that it is very difficult to ensure good metal-to-metal contact between the core layer 611 and the housing rear wall 601 due to surface imperfections on the contacting surfaces. And, although the TIM paste is used to improve the heat transfer, the TIM typically has a much lower heat transfer coefficient as compared to the metal that forms the housing.

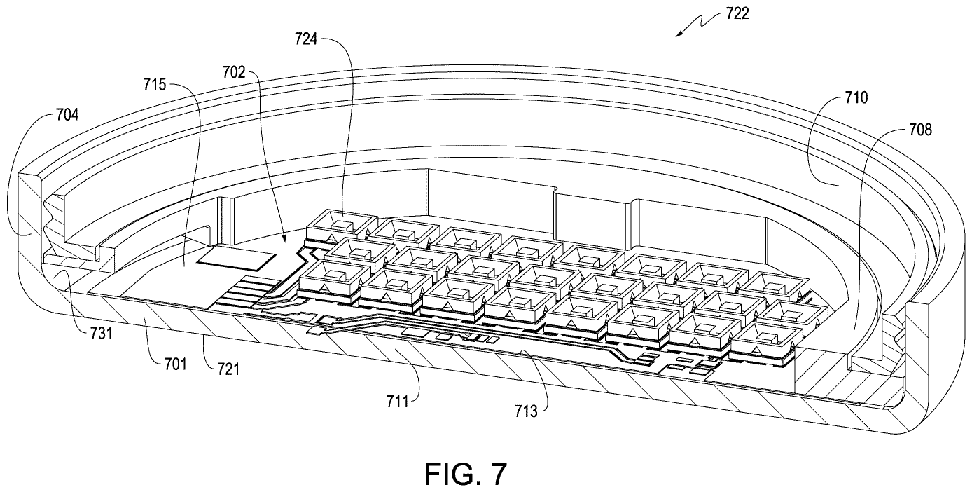

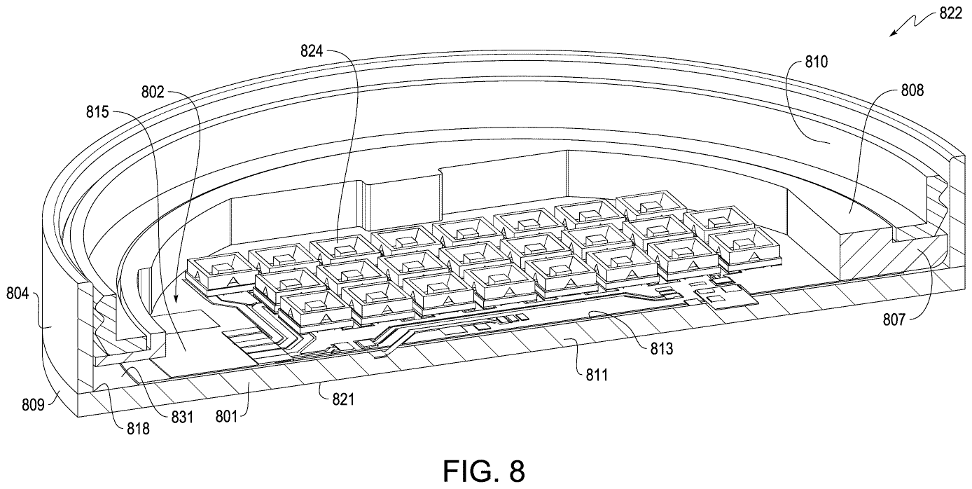

FIG. 7 illustrates one embodiment of a UV-LED assembly according to this invention. FIG. 7 is a cross-sectional view taken through the center of the light source unit 722. FIG. 7 includes a similar bushing 708 and sealing ring 710 as in the FIG. 6 embodiment that support a transparent quartz window, which is secured by a retaining ring. In FIGS. 7 and 8 the quartz window and the retaining ring are not illustrated, but these structures can be configured similarly as shown and described in connection with FIG. 6. The bushing 708 can be rigid and made out of a plastic such as a fluoropolymer. The sealing ring 710 can be made from an elastomer, for example, and could be a circular O-ring. In this embodiment, a MCPCB or CCPCB 702 is included in the light source unit 722, and includes a core layer (either metal or thermally conductive ceramic) 711, dielectric layer 713, trace layer 715 (including a metal such as copper), and LED array 724 that is electrically connected to the trace layer 715.

In this embodiment, the core layer 711 is integrated into the housing of the light source unit 722 and forms the rear wall 701 and sidewall 704. Thus, the exterior surface 721 of rear wall 701 and side wall 704 is part of the MCPCB or CCPCB core layer 711. When the UV-assembly is fixed in a reactor that is used to treat water or other fluids, the exterior surface 721 will be in direct physical contact with the fluid that is being treated in the reactor. The core layer 711 of the PCB is thereby “wetted” by the fluid and heat is transferred directly from the core layer 711 without having to also transfer heat through a separate housing layer and a TIM layer. This eliminates the contact resistance between the PCB and the TIM, and between the TIM and the housing material. This design also eliminates the thermal resistance of a separate housing layer.

In this case the walls 701, 704 will form a housing that is directly or indirectly sealed to the transparent quartz window to form a water-tight space that houses the LEDs 712. As discussed above, a threaded retaining ring can be fastened over the quartz window so that it is clamped down on the bushing 708 and sealing ring 710. Alternatively, flanges or crimps can be formed on the top surface 722 of sidewall 704 that are bent to hold the transparent quartz window in place. Fingers or notches can be formed in the retaining ring (NOT SHOWN) that interface with complementary features on the side wall 704 to fasten the retaining ring to the sidewall to retain the quartz window. Various other methods, such as snap rings, could be used to hold the quartz window in place.

In some aspects, the housing of the light source unit 722 can be formed by stamping or bending the core layer 711 so that a periphery of the core layer 711 is bent to extend transversely or substantially orthogonally to a central region of the core layer 711. The transversely extending portion will ultimately form sidewall 704 and the central region will ultimately form the rear wall 701. If the core layer 711 is ceramic, the core layer 711 could be molded or formed into this shape without bending. The dielectric layer and trace layer and components could be provided on the upper surface 731 of core layer 711 before forming, or could be provided afterward. Once the core layer 711 is formed into the desired shape, the dielectric layer 713 and trace layer 715 can be respectively formed on the upper surface 731 in a central region of the core layer 711, and the LED array 724 can be electrically connected to the trace layer 715. A seal can be formed between the peripheral region of the core layer 711 and a transparent window, as described above, to provide a water-tight cavity that houses the LED array 724. Alternatively, the MCPCB or CCPCB 702 can be pre-formed where the dielectric layer 713, trace layer 715, and optionally the LED array 725 are formed, and a peripheral portion of the upper surface 731 of the core layer is free of the dielectric layer 713 and the trace layer 715 (i.e., such that the core layer 711 has a longest dimension that is larger than either of layers 713, 715). The peripheral portion of the core layer 711 can then be stamped or bent into the desired shape to form the housing side wall 704, and the light source unit 722 can be subsequently assembled.

FIG. 8 illustrates another embodiment of a UV-LED assembly according to this invention. FIG. 8 is a cross-sectional view taken through the center of the light source unit 822. FIG. 8 includes a similar bushing 808 and sealing ring 810 as in the above embodiments that supports a transparent window. In this embodiment, a MCPCB or CCPCB 802 is included in the light source unit 822, and includes a core layer (either metal or thermally conductive ceramic) 811, dielectric layer 813, trace layer 815 (including a metal such as copper), and LED array 824 that is electrically connected to the trace layer 815. In this embodiment, an upper surface 831 of the core layer 811 is free of the dielectric layer 813 and the trace layer 815. A side wall member 804 can be joined to a portion of the core layer 811 to form a seal 818 therewith. FIG. 8 shows a bottom surface of sidewall 804 being joined to the upper surface 831 of the core layer 811, but alternatively an inner surface of side wall member 804 could be joined to side surface 809 of the core layer. The seal 818 can be formed with an adhesive, or sealing paste, or with an elastomeric O-ring, or by stir welding, for example. In this embodiment, the retaining ring (e.g., as shown in FIG. 6) could be formed as part of sidewall 804.

As in the FIG. 7 embodiment, when the UV-assembly is fixed in a reactor that is used to treat water or other fluids, the exterior surface 821 will be in direct physical contact with the fluid that is being treated in the reactor, or with a cooling fluid. The core layer 811 of the PCB thus is “wetted” by the fluid and heat is transferred directly from the core layer 811 without having to also transfer heat through a separate housing layer and a TIM layer.

The FIGS. 7 and 8 embodiments illustrate the application of the invention into a UV-LED assembly with a puck-shaped light source unit. But the light source unit can be formed into any shape in which the core layer of the MCPCB or CCPCB can constitute at least a portion of an external surface of the UV-LED assembly so that a cooling fluid can directly contact and cool the core layer of the MCPCB or CCPCB.

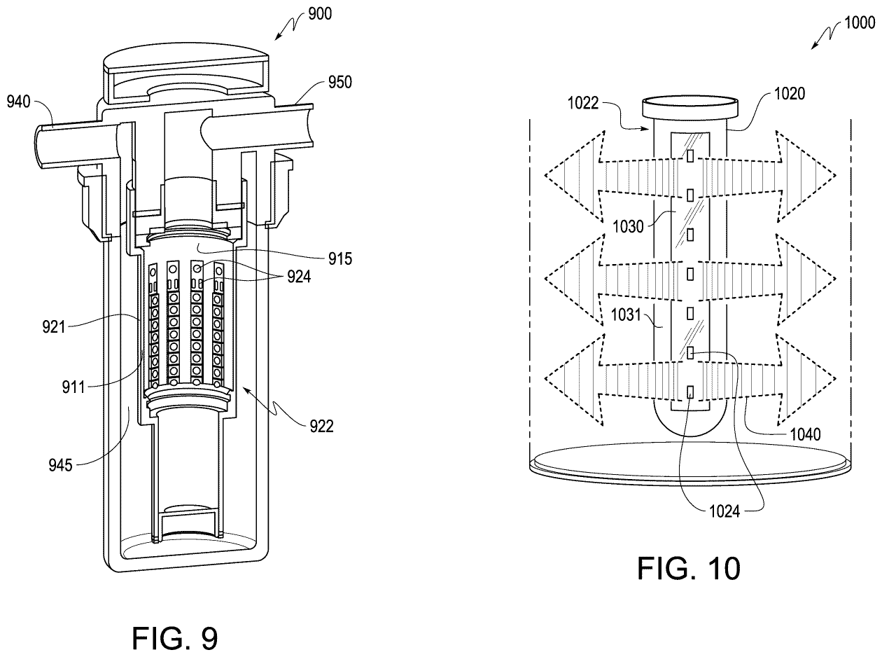

FIG. 9 illustrates a cross-section of a UV treatment reactor 900 in which the light source unit 922 has a substantially tubular shape. In this case, the MCPCB or CCPCB is formed as part of, or the entirety of a tubular shape so that the trace layer 915 and LEDs 924 face radially inwardly, and exterior surface 921 of the core layer 911 faces radially outwardly. Fluid can enter reactor at inlet 940, flow downwardly along channel 945 while contacting and cooling exterior surface 921, and can then flow upwardly through the light source unit 922 where it is irradiated with UV light before exiting the reactor at exit 950. Similar configurations could also be implemented where the fluid flows in the reverse direction, i.e., downwardly through the light source unit 922 in FIG. 9. As in the FIG. 7 embodiment, the shape of the core layer 911 can be formed either before or after forming the dielectric layer and the trace layer 915.

FIG. 10 illustrates a cross-section of a UV treatment reactor1000 in which the light source assembly has a light source unit 1022 with a cylindrical or rod shape. In this case, the MCPCB or CCPCB could be formed so that a portion of the upper surface 1031 of the core layer is formed to be free of the dielectric layer and the trace layer. The core layer is formed into a rod shape so that the upper surface 1031 of the core layer faces radially outwardly and forms and exterior surface of the light source assembly. Portions of the upper surface 1031 that include the dielectric layer, trace layer, and LEDs 1024 can be covered and sealed, e.g., with a transparent window 1030. Water or other fluid to be treated can enter into the reactor and can be circulated within the reactor in various ways so that the fluid is treated with UV light generated from UV-LEDs 1024 while contacting the upper surface 1031 of the core layer to cool the light source assembly.

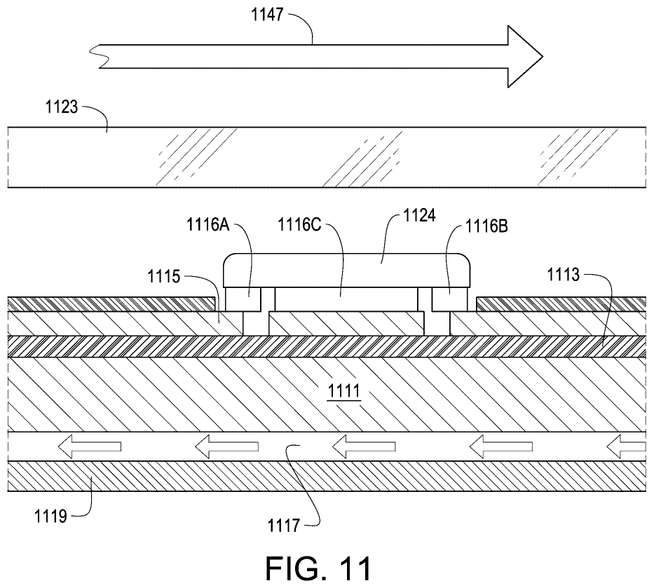

FIG. 11 schematically illustrates a MCPCB or CCPCB that has a separate cooling flow 1117 that is distinct from the main flow 1147. In this system, LED 1124 emits light into a treatment zone (above transparent window 1123 in FIG. 11) in which the main flow 1147 of fluid is treated. As described above in connection with FIG. 4, heat is dissipated from the LED 1124 through solder pads 1116A, 1116B, and 1116C, trace layer 1115, dielectric layer 1113, and core layer 1111. Other PCB arrangement such as those illustrated in connection with FIGS. 5A and 5B can also be used. The cooling flow 1117 is formed by a channel that is bounded by the bottom surface of the core layer 1111 and a fluid retaining wall 1119, and the cooling fluid in cooling flow 1117 directly contacts the bottom surface of the core layer 1111 to transfer heat from the device. The cooling flow 1117 can include the same cooling fluid that flows in the main flow or a different fluid from the main flow, and can receive the cooling fluid at an inlet and discharge it at an outlet.

Although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is

1. A reactor for treating a fluid with UV radiation, the reactor comprising:
(i) a reactor vessel through which the fluid flows; and
(ii) a UV-LED assembly that is arranged in the reactor vessel and comprises:
(a) a printed circuit board that includes a plurality of UV-LEDs and a metal core or ceramic core substrate, and
(b) a transparent window arranged on the UV-LED assembly so that UV light from the UV-LEDs passes through the transparent window and into the fluid flowing through the reactor vessel,
wherein a surface of the metal or ceramic core substrate constitutes a surface of the UV-LED assembly that is configured to be in direct contact with a cooling fluid.

2. The reactor of claim 1, wherein the cooling fluid is the same as the fluid flowing through the reactor vessel.

3. The reactor of claim 1, wherein the cooling fluid includes a flow of fluid that is separate from the fluid flowing through the reactor vessel.

4. The reactor of claim 1, wherein the printed circuit board includes a dielectric layer that is formed on a portion of a first surface of the metal core or ceramic core substrate.

5. The reactor of claim 4, wherein a second surface of the metal core or ceramic core substrate that is opposite the first surface is the surface that forms the surface that is configured to be in direct contact with a cooling fluid.

6. The reactor of claim 1, wherein the surface of the metal or ceramic core substrate constitutes an exterior surface of the UV-LED assembly.

7. The reactor of claim 4, wherein the first surface has a region that is free of the dielectric layer.

8. The reactor of claim 7, wherein the UV-LED assembly includes a housing wall that is joined to the region of the first surface that is free of the dielectric layer.

9. The reactor of claim 8, wherein the housing wall forms a seal with the transparent window so that the plurality of UV-LEDs are contained in water tight housing.

10. The reactor of claim 1, wherein the metal core or ceramic core substrate includes a peripheral region and a central region, and the peripheral region is bent relative to the central region.

11. The reactor of claim 10, wherein the peripheral region and/or the central region of the metal core or ceramic core substrate forms a seal with the transparent window so that the plurality of UV-LEDs are contained in a water-tight housing.

12. The reactor of claim 10, wherein the central region of the metal core or ceramic core substrate extends in a plane parallel to the transparent window, and the peripheral region extends orthogonal to the central region.

13. The reactor of claim 1, wherein the UV-LED assembly comprises a metal core printed circuit board having a metal core substrate that is a single layer.

14. The reactor of claim 1, wherein the UV-LED assembly comprises a ceramic core printed circuit board having a ceramic core substrate that is a single layer.

15. The reactor of claim 1, wherein the UV-LED assembly is free of a thermal interface material.

16. The reactor of claim 1, where the UV-LED assembly has a portion containing the plurality of LEDs that is shaped like a cylinder, a rod, or a tube.

17. A method of treating a fluid comprising:
passing the fluid through the reactor of claim 1,
generating UV light from the plurality of UV-LEDs, and
transmitting the UV light into the fluid.

18. A UV-LED assembly comprising:
(a) a printed circuit board that includes a plurality of UV-LEDs and a metal core or ceramic core substrate, and
(b) a transparent window arranged on the UV-LED assembly so that UV light from the UV-LEDs passes through the transparent window,
wherein a surface of the metal core or ceramic core substrate constitutes an exterior surface of the UV-LED assembly.

19. The UV-LED assembly of claim 18, wherein the transparent window is sealed to the metal core or ceramic core substrate to form a water-tight cavity that contains the plurality of UV-LEDs.

20. The UV-LED assembly of claim 19, wherein the assembly further comprises a housing wall that is sealed to the metal core or ceramic core substrate to form the water-tight cavity.
